(19) United States
(12) Patent Application Publication
(10) Pub. No.: US 2024/0092720 A1
(43) Pub. Date: Mar. 21, 2024

(54) ANTI-FOULING COMPOSITION AND USES THEREOF

(71) Applicant: SPECIALTY OPERATIONS FRANCE, Lyon (FR)

(72) Inventors: Jing Jiang, Shanghai (CN); David Vanzin, Franklin, TN (US); Rodolphe Sapey-Triomphe, Serezin-Du-Rhone (FR); Shujing Cheng, Shanghai (CN); Lars Fischer, Vienne (FR)

(73) Assignee: SPECIALTY OPERATIONS FRANCE, Lyon (FR)

(21) Appl. No.: 18/264,213

(22) PCT Filed: Feb. 4, 2021
(86) PCT No.: PCT/CN2021/075286

§ 371 Date: Aug. 3, 2023

(51) Int. Cl. C07C 57/04; C07C 51/50

(52) U.S. Cl. CPC C07C 57/04; C07C 51/50

(57) ABSTRACT

An anti-fouling composition, and uses thereof in the industrial preparation of ethylenically unsaturated monomers comprising at least one heteroatom, such as acrylic monomers are disclosed.

### TECHNICAL FIELD

The present invention relates to the field of the industrial preparation of ethylenically unsaturated monomers comprising at least one heteroatom, such as acrylic monomers. In particular, the present invention relates to an anti-fouling composition and uses thereof.

### BACKGROUND

Ethylenically unsaturated monomers have the property of undergoing spontaneous polymerization, more particularly under the effect of heat. This polymerization proves disruptive during monomer preparation steps, particularly during the manufacture, purification, and storage of said monomers. Unless it is prevented, it gives rise to detrimental yield losses. Frequent stops for plant maintenance are then necessary, in order to remove the deposits formed, and the production capacities are therefore reduced as a result, and in that case an extra production cost is incurred.

For example, the preparation of acrylic acid or acrylic esters from acrylic acid conventionally requires a distilling operation for the purposes of separating, concentrating, or purifying the monomer in question. Acrylic acid, however, when taken to a higher temperature, like that required for a distillation, exhibits a tendency to polymerize. The polymeric material thus formed, in liquid phase or vapor phase, then undergoes deposition on the various pieces of equipment employed for the process, and more particularly on the walls of the columns, on the plates, and on the condensers. The result of this is fouling of this equipment, which leads to the above described production losses and increased operational costs.

Solutions have already been proposed to combat this phenomenon of premature polymerization, particularly during distillations. It is in this vain that polymerization inhibitors are commonly employed. These are, most frequently, phenol derivatives, amine derivatives, thiazine derivatives, nitroso derivatives, and N-oxyl derivatives. Other polymerization inhibitors are also known, such as metal salts or else, particularly, quinone derivatives.

Preference, among these classes of polymerization inhibitors, is generally given to the use, on account of their enhanced effectiveness, of phenol derivatives such as, particularly, hydroquinone (HQ), monomethyl hydroquinone ether (MEHQ), 2,6-di-tert-butyl-paracresol (BHT), and 2,4-dimethyl-6-tert-butylphenol (Topanol A); amine derivatives such as phenylenediamine; thiazine derivatives such as, particularly, phenothiazine (PTZ) or methylene blue; nitroso compounds such as N-nitrosodiphenylamine; N-oxyl derivatives such as, for example, 2,2,6,6-tetramethyl-1-piperidine N-oxyl (TEMPO) and its derivatives: 4-hydroxy TEMPO (OH-TEMPO), 4-methoxy TEMPO, 4-oxo TEMPO, or 4-amino TEMPO; metal salts such as, for example, iron sulfate or a copper salt; or quinone derivatives such as para-benzoquinone or di-tert-butyl benzoquinone.

With a concern to have polymerization inhibitor systems of increased effectiveness available, moreover, proposals have already been made to combine some of the aforementioned inhibitors.

Accordingly, EP 765 856 describes the combined use of an N-oxyl derivative-type polymerization inhibitor with a phenol derivative-type polymerization inhibitor, with the aim of stabilizing acrylic acid during distillation or else during transport. EP 1 273 565 discloses the use of hydroquinone, Topanol A, BHT, and phenothiazine, either alone or in combination, for preventing the risk of polymerization in the distillation column during the synthesis and purification of (meth) acrylic anhydride.

Likewise in the context of the production of (meth) acrylic anhydrides, EP 1 805 128 describes the use of a polymerization inhibitor selected from the group consisting of (a) metal salts of thiocarbamic acid or dithiocarbamic acid and mixtures thereof with a phenol derivative or with phenothiazine and its derivatives, and (b) N-oxyl compounds in a mixture with 2,6-di-tert-butyl 4-methylphenol (BHT) alone or in the presence of 2,4-dimethyl 6-tert-butylphenol (Topanol A). U.S. Pat. Nos. 6,540,881 B1 and 7,029,556 B1 detail these types of systems in the context of acrylic acid.

US 2006/0142613 describes the combined use of a phenol derivative and a copper-based polymerization inhibitor.

While these polymerization inhibitor systems do prove to be actually more satisfactory, they nevertheless remain capable of improvement. The reason is that, an inhibitor system combining a phenol derivative such as hydroquinone (HQ) with a thiazine derivative such as phenothiazine (PTZ) does not constitute a complete escape from the phenomenon of polymerization.

Furthermore, proposals have already been made, for enhancing the production of certain ethylenically unsaturated monomers, to add dispersants so as to disperse the fouling material originating from the unwanted polymerization and to prevent their deposition on plant surfaces, so as to obviate excessive and expensive maintenance operations.

Accordingly, U.S. Pat. No. 5,650,072 proposes the addition of a naphthalene sulfonate-formaldehyde condensate during the production of acrylonitrile in order to control fouling on the walls of industrial plants. Still within the context of acrylonitrile production, U.S. Pat. No. 8,067,629 describes the use of a styrene sulfonate polymer as a dispersant preventing the deposition of unwanted residues on the walls of industrial plants. Lastly, U.S. Pat. No. 7,880,029 describes the employment of N-alkylsuccinimide during the formation of acrylic monomers.

WO2017041204A1 provides an inhibitor composition comprising para-methoxyphenol (PMP) and pyrocatechol (PC), as well as at least one ancillary polymerization inhibitor.

There still exists in the present field a wish to make further improvements to the inhibitor composition as well as to the dispersant, for various aspects including the performance of polymerization inhibition and anti-fouling, and/or cost effectiveness thereof.

### BRIEF DESCRIPTION OF THE INVENTION

The inventors of the present invention unexpectedly discovered that certain types of dispersants demonstrated better anti-fouling performance than others when used together with inhibitors. The inventors also unexpectedly discovered that specific combinations of inhibitors may achieve synergic effects in inhibiting the unwanted polymerization of monomers.

One subject matter of the present invention is a new anti-fouling composition comprising at least one polymerization inhibitor (a polymerization-inhibiting composition) and at least one specific dispersant.

Another subject matter of the present invention is a method for purifying a monomer composition, which comprises at least one ethylenically unsaturated monomer comprising at least one heteroatom, comprising a distillation step of the monomer composition in the presence of the anti-fouling composition of the present invention.

Yet another subject matter of the present invention is the use of the anti-fouling composition of the present invention, to limit and/or prevent fouling of industrial equipment(s) used during reaction, distillation, purification, storage, transportation and/or handling.

### BRIEF DESCRIPTION OF THE FIGURES

FIG. 1 shows a schematic diagram of the distillation apparatus used in the examples.

### DETAILED DESCRIPTION OF THE INVENTION

For convenience, before further description of the present disclosure, certain terms employed in the specification, and examples are collected here and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

Throughout the description, including the claims, the term “comprising one” should be understood as being synonymous with the term “comprising at least one”, unless otherwise specified, and “between” should be understood as being inclusive of the limits. It is specified that, in the continuation of the description, unless otherwise indicated, the values at the limits are included in the ranges of values which are given.

The expression “comprise” should be understood as including equally “consist of” or “consist substantively of”.

It should be noted that in specifying any numerical range, such as a range of contents or ratios, any particular upper limit can be associated with any particular lower limit, any two particular numerical values can be associated together to form a new numerical range.

If not specified otherwise, a percentage or ppm content is on weight basis.

Polymerization-Inhibiting Composition

In the present invention, the at least one polymerization inhibitor forms a polymerization-inhibiting composition, and the at least one polymerization inhibitor is selected from the group consisting of hydroquinone (HQ), phenothiazine (PTZ), monomethyl hydroquinone ether (MEHQ), TEMPO and its derivatives, and copper salts.

Hydroquinone (HQ) is normally a white needle-like crystal, flammable, soluble in hot water, ether and ethanol, and slightly soluble in benzene.

In the present invention, HQ can be used in solid form, or in a solubilized form, as long as the solvent is compatible with the monomer composition, with which the polymerization-inhibiting composition is used.

Phenothiazine (PTZ) is normally a yellow powder, or in the form of yellow prills, insoluble in petroleum ether, chloroform and water, and soluble in ether and hot acetic acid.

In the present invention, PTZ can be used in solid form, or in a solubilized form, as long as the solvent is compatible with the monomer composition, with which the polymerization-inhibiting composition is used.

Monomethyl hydroquinone ether (MEHQ) is normally a white flaky crystal, easily soluble in alcohol, ether, acetone, toluene and ethyl acetate, and slightly soluble in water.

In the present invention, MEHQ can be used in solid form, in molten/liquid form, or in a solubilized form, as long as the solvent is compatible with the monomer composition, with which the polymerization-inhibiting composition is used.

In the present invention, TEMPO is the short term for 2,2,6,6-tetramethyl-1-piperidine N-oxyl (TEMPO). TEMPO derivatives include but not limited to 4-hydroxy TEMPO (OH-TEMPO), 4-methoxy TEMPO, 4-oxo TEMPO, or 4-amino TEMPO.

In the present invention, the copper salt is at least one selected from the group consisting of (basic) copper sulfate, copper acetate, copper nitrate, (basic) copper carbonate, copper chloride, copper naphthenate, copper benzoate, copper stearate, copper acrylate and CDBC (copper dibutyldithiocarbarmate). As is conventional to a skilled person, (basic) copper carbonate represents copper carbonate and/or basic copper carbonate.

As is conventional to a skilled person, when applicable, the crystals of one or more of the above copper salts may comprise a certain amount of crystalized water. For example, basic copper carbonate can be used in its hydrated form with a formula of CuCO₃·Cu(OH)₂·xH2O, the specific amount of x is conventional to a skilled person.

In one embodiment of the present invention, the polymerization-inhibiting composition comprises or consists of HQ and PTZ.

In one embodiment of the present invention, the polymerization-inhibiting composition comprises or consists of HQ, PTZ and MEHQ.

In one embodiment of the present invention, the polymerization-inhibiting composition comprises or consists of HQ, PTZ and at least one copper salt, such as, basic copper carbonate and/or CDBC.

In one embodiment of the present invention, the polymerization-inhibiting composition comprises or consists of HQ, PTZ, MEHQ and at least one copper salt, such as, basic copper carbonate and/or CDBC.

In one embodiment of the present invention, the polymerization-inhibiting composition comprises or consists of TEMPO and/or its derivatives.

In one embodiment of the present invention, the polymerization inhibiting composition comprises or consists of TEMPO and/or its derivatives, as well as HQ and/or PTZ.

In one embodiment of the present invention, the polymerization-inhibiting composition comprises or consists of TEMPO and/or its derivatives, as well as MEHQ and/or at least one copper salt, such as basic copper carbonate and/or CDBC.

In one embodiment of the present invention, the content of HQ based on the total weight of the polymerization-inhibiting composition is in the range of 20 wt %-70 wt %, preferably 30 wt %-60 wt %, and more preferably 40 wt %-50 wt %.

In one embodiment of the present invention, the content of PTZ based on the total weight of the polymerization-inhibiting composition is in the range of 20 wt %-70 wt %, preferably 30 wt %-60 wt %, and more preferably 40 wt %-50 wt %.

In one embodiment of the present invention, the content of TEMPO and/or its derivatives based on the total weight of the polymerization-inhibiting composition is in the range of 1 wt %-100 wt %, preferably 30 wt %-90 wt %, and more preferably 40 wt %-80 wt %.

In one embodiment of the present invention, the content of the at least one copper salt based on the total weight of the polymerization-inhibiting composition is in the range of 1 wt %-30 wt %, preferably 3 wt %-20 wt %, and more preferably 5 wt %-10 wt %.

In one embodiment of the present invention, the content of MEHQ based on the total weight of the polymerization-inhibiting composition is in the range of 0.1 wt %-40 wt %, preferably 0.1 wt %-30 wt %, and more preferably 0.2 wt %-20 wt %, such as 0.8 wt %, 5 wt % or 10 wt %.

In one embodiment of the present invention, the weight ratio of HQ:PTZ is in the range of (0.4-2.5):1, preferably (0.8-1.5):1, and more preferably (0.9-1.1):1.

In one embodiment of the present invention, the polymerization-inhibiting composition is free of CDBC (copper dibutyldithiocarbarmate).

In one embodiment of the present invention, the polymerization-inhibiting composition is used in a monomer composition, which comprises at least one ethylenically unsaturated monomer comprising at least one heteroatom, which will be described in further detail below.

In one embodiment of the present invention, the components comprised in the polymerization-inhibiting composition are used at the same time as a blend. In one embodiment of the present invention, in the blend all the components are in solid form, or at least one of the components are in other forms than solid, such as in solubilized form (with at least one solvent) or in liquid form (without a solvent). For example, MEHQ can be used in an aqueous solution, while HQ, PTZ and copper salt be used in solid form.

In one embodiment of the present invention, at least one of the components comprised in the polymerization-inhibiting composition are used at a different time and/or location from that of the other components.

Anti-Fouling Composition

In one embodiment of the present invention, the anti-fouling composition comprises at least one polymerization inhibitor conventionally used in the field and at least one dispersant.

In the present invention, the term “dispersant” is used to name a component, preventing deposits (particularly of polymers) and the fouling of industrial equipment, particularly of purifying equipment.

In one embodiment of the present invention, the at least one dispersant works compatibly or synergistically with the inhibitor(s) to prevent deposits (particularly of polymers) and the fouling of industrial equipment, particularly of purifying equipment.

The classes of dispersants which may be employed in the context of the present invention are particularly as follows:
sulfonates, such as styrenesulfonate, naphthalenesulfonate, for instance SUPRAGIL MNS/90, which is a polyalkylnaphthalenesulfonate sold by Solvay,
alkyl ether sulfates, such as ammonium and sodium salt of alkyl ether sulfate,
esters, such as the methyl ester of salicylic acid, also called methyl salicylate, sucrose ester,
succinimides, such as polyisobutenylsuccinimide,
tristyrylphenols, such as ethoxylated TSP and tristyrylphenol phosphate ethoxylate,
acrylates, such as ethyl methacrylate, ethoxy methacrylate, and 2-ethylhexyl acrylate,
amides, such as dimethylamides, acrylamides such as N-tert-butylacrylamide or N-(butoxymethyl) methacrylamide, alkyl alcohol amide
amines, such as isopropylhydroxyl amine and Mannich bases,
imidazoline, such as 1-aminoethyl-2-C17 alkylene-2-imidazoline,
phenates, such as sulfurized calcium alkylphenate,
phosphates, such as sodium polyphosphates such as the tripolyphosphate, phosphate esters, and ethoxylated phosphate esters,
EO/PO block copolymer
sorbitan esters and ethoxylated sorbitan esters
phenols, alkyl phenols, alkyl aryl phenol and their ethoxylates, propoxylates or alkoxylates
linear and branched alcohols and their ethoxylates, propoxylates or alkoxylates
linear and branched acids and their ethoxylates, propoxylates or alkoxylates
alkyl and aryl oils and their ethoxylates, propoxylates or alkoxylates.
and mixtures thereof.

In one embodiment of the present invention, the at least one dispersant is particularly selected from the group consisting of alkyl ether sulfates, sulfonates, half esters of sulfosuccinic acid, sulfates, polyoxyethylenes, sorbitan and ethoxylated sorbitan esters, fatty alcohol ethoxylates and ethoxylate/propoxylate block copolymers.

In one embodiment of the present invention, the at least one dispersant is more particularly selected from the group consisting of sulfonates, half esters of sulfosuccinic acid, sulfates, sorbitan and ethoxylated sorbitan esters, fatty alcohol ethoxylates and ethoxylate/propoxylate block copolymers.

In one embodiment of the present invention, the at least one dispersant can be used in solid form, in liquid form, or in a solubilized form, as long as the solvent is compatible with the monomer composition, with which the anti-fouling composition is used.

In one embodiment of the present invention, the content of the at least one dispersant based on the total weight of the anti-fouling composition is in the range of 1 wt %-50 wt %, preferably 1.5 wt %-40 wt %, more preferably 2 wt %-30 wt %, still more preferably 2 wt %-20 wt %, such as 10 wt % or 15 wt %.

In one embodiment of the present invention, the anti-fouling composition comprises or consists of:
a first portion comprising the at least one dispersant,
a second portion comprising hydroquinone (HQ), and
a third portion comprising phenothiazine (PTZ).

In one embodiment of the present invention, the anti-fouling composition comprises or consists of:
a first portion comprising the at least one dispersant, and
a fourth portion comprising TEMPO and/or its derivatives.

In one embodiment of the present invention, the first portion is provided in an aqueous form.

In one embodiment of the present invention, the second portion is provided in a solid form, which is dissolved in water prior to use, or provided in an aqueous form.

In one embodiment of the present invention, the third portion is provided in a solid form, which is dissolved in at least one suitable solvent prior to use, or provided in a solution form comprising at least one suitable solvent, such as toluene.

In one embodiment of the present invention, the fourth portion is provided in a solid form, which is dissolved in at least one suitable solvent prior to use, or provided in an aqueous form.

In one embodiment of the present invention, the first portion further comprises MEHQ.

In one embodiment of the present invention, the first portion comprises 15-99 wt % of the at least one dispersant and 1-10 wt % of MEHQ. In one embodiment of the present invention, the first portion is an aqueous solution comprising 15-99 wt % of the at least one dispersant, 1-10 wt % of MEHQ and balance of water. In one embodiment of the present invention, the first portion comprises for example 50 wt %, 60 wt %, 70 wt %, 80 wt % or 90 wt % of the at least one dispersant and/or for example 3 wt %, 5 wt %, 7 wt % or 9 wt % of MEHQ. In one embodiment of the present invention, the first portion consists of the at least one dispersant and MEHQ.

In one embodiment of the present invention, the second portion further comprises (basic) copper carbonate.

In one embodiment of the present invention, the third portion further comprises CDBC.

In one embodiment of the present invention, the fourth portion further comprises at least one selected from the group consisting of HQ, PTZ, (basic) copper carbonate and CDBC.

In one embodiment of the present invention, the first portion and the second portion are provided in a single aqueous solution.

In one embodiment of the present invention, the first portion and the fourth portion are provided in a single aqueous solution.

In one embodiment of the present invention, the second portion and the third portion are provided as in the solid state, which are preferably dissolved together in at least one suitable solvent prior to use.

In one embodiment of the present invention, the anti-fouling composition is used in a monomer composition, with a content of the anti-fouling composition in the monomer composition in a range of 10-5000 ppm by weight, preferably 100-4000 ppm, more preferably 1000-3000 ppm, still more preferably 1500-2500 ppm.

In one embodiment of the present invention, the monomer composition comprises at least one ethylenically unsaturated monomer comprising at least one heteroatom, which will be described in further detail below.

In one embodiment of the present invention, the components comprised in the anti-fouling composition are used at the same time as a blend. In one embodiment of the present invention, at least one of the components are in other forms than that of the dispersant. For example, MEHQ and the dispersant can be used in an aqueous solution, while HQ, PTZ and copper salt be used in a solid form.

In one embodiment of the present invention, at least two of the components comprised in the anti-fouling composition are used at the same time and/or location.

In one embodiment of the present invention, at least one of the components comprised in the anti-fouling composition are used at a different time and/or location from that of the other components.

Ethylenically Unsaturated Monomers

In the context of the present invention, an ethylenically unsaturated monomer is a monomer comprising at least one ethylenic unsaturation. A heteroatom is any atom that is not carbon or hydrogen. Typical heteroatoms are nitrogen, oxygen, sulphur, phosphorus, chlorine, bromine, and iodine.

Ethylenically unsaturated monomers comprising at least one heteroatom comprise, in particular, halogenated unsaturated monomers, acrylic monomers, unsaturated acrylic resins, unsaturated amides, unsaturated ethers, vinylpyridines, and vinyl acetate.

Halogenated unsaturated monomers include vinyl chloride, chloroprene, vinylidene chloride, vinylidene fluoride, vinyl fluoride and mixtures thereof.

Acrylic monomers include unsaturated acids typified by acrylic acid (AA), methacrylic acid (MAA), and crotonic acid; acrylates typified by methyl acrylate, ethyl acrylate, n-butyl acrylate, 2-ethylhexyl acrylate, hydroxyethyl acrylate, hydroxypropyl acrylate, dimethylaminomethyl acrylate, or any other acrylate derivative; methacrylates (MA), typified by methyl methacrylate, ethyl methacrylate, butyl methacrylate, lauryl methacrylate, dimethylaminoethyl methacrylate, and stearyl methacrylate; acrylonitrile (ACN), acrolein, acrylic anhydride, methacrylic anhydride and mixtures thereof.

Unsaturated acrylic resins include acrylated epoxy resins and polyethylene glycol diacrylate.

Unsaturated amides include acrylamide, N, N-dimethylacrylamide, methylenebisacrylamide, and N-vinylpyrrolidone.

Unsaturated ethers include vinyl methyl ether.

Other ethylenically unsaturated monomers comprising at least one heteroatom include, further, vinyl acetate, diethyl vinylphosphonate, styrenesulfonic acids and sodium styrenesulfonate.

In one embodiment of the present invention, the ethylenically unsaturated monomers comprising at least one heteroatom are selected from acrylic monomers.

In one embodiment of the present invention, the ethylenically unsaturated monomers comprising at least one heteroatom are selected from the group consisting of acrylic acid, methacrylic acid, methyl acrylate, ethyl acrylate, methyl methacrylate, ethyl methacrylate, and acrylonitrile.

In one embodiment of the present invention, the monomer composition comprises a solvent or a solvent mixture. The solvent or solvent mixture of the monomer composition is selected from one or more of the group consisting of:
water;
alcohols including methanol, ethanol, n-propanol, isopropanol, n-butanol and isobutanol etc.;
ketones including acetone, butyl ketone, cyclopentanone and cyclohexanone etc.;
ethers including ethylene glycol monobutyl ether, diethylene glycol monomethyl ether (DEGME) and diethylene glycol monobutyl ether (DEGBE) etc.; and
aromatic hydrocarbons including benzene, toluene, ethylbenzene, and xylene etc.

The increase in the rate of use of equipment via the polymerization-inhibiting activity and/or the anti-fouling effect may be desired in all industrial stages involving the use of ethylenically unsaturated monomer(s) comprising at least one heteroatom. Mention may be made more particularly of the stages of purification or else the stages of manufacture, storage, transport, handling, etc.

In the case of use in a purification step, the purifying equipment, in particular a distillation equipment, for which the present invention proposes, more particularly, to control fouling of their walls and their internals may be, in particular, the distillation columns themselves, scrubbing columns, absorption columns, or else all of the peripheral equipment such as condensers, pumps, boilers, phase separators, and the associated piping. In one embodiment of the present invention, the distillation column is more particularly an azeotropic distillation column. The polymerization-inhibiting composition or the anti-fouling composition described in the context of the present invention may also be used in liquid/liquid extractions.

The polymerization-inhibiting composition or the anti-fouling composition described in the present invention may be added entirely conventionally to the monomer composition. It is possible, moreover, to envisage the continuous or else repeated addition of said compositions over time, at one or more points of introduction. Furthermore, the addition of HQ, PTZ, copper salt, MEHQ and dispersants, and optional additives may be simultaneous or separate.

As mentioned above, the present invention is also directed to a process for preparing ethylenically unsaturated monomers comprising at least one heteroatom, particularly acrylic monomers, employing a distillation of said monomers, wherein said monomers are distilled in the presence of the polymerization-inhibiting composition of the present invention, or the anti-fouling composition of the present invention.

The distillation temperatures at which equipment fouling is most particularly observed are commonly between 30 and 300° C., more particularly between 40 and 200° C. and, for example, between 50 and 90° C., particularly at a pressure of between 0 and 3 bar in absolute pressure, more particularly between 0.1 and 2 bar, and, for example, between 0.3 and 1.5 bar in absolute pressure, and in the presence of oxygen.

The preset invention proves especially advantageous for these operating conditions. The polymerization-inhibiting composition or the anti-fouling composition of the present invention may be in a solid form after having been shaped by any of various methods: powders, crystals, flakes, prills, without limitation, and obtained by mixing or by co-generation of solids (crystallizing, flaking, atomizing, prilling, granulated, pelletizing, etc.).

The composition may alternatively be formulated with a solvent, and/or may be formulated with an amount of monomer in accordance with the monomer intended for treatment according to the invention.

The polymerization-inhibiting composition or the anti-fouling composition of the present invention may further comprise one or more additives selected from detergents other than the dispersants, antioxidants, antifoams, rust inhibitors, corrosion inhibitors, and surfactants distinct from the dispersants in accordance with the invention. The detergents distinct from the dispersants are advantageously selected from salicylates, phenates different from the dispersants, and sulfonates different from the dispersants. The antioxidants are advantageously selected from amines different from the dispersants, and derivatives of phenol. The antifoams are advantageously selected from silicones and acrylates. The rust inhibitors are advantageously selected from amines different from the dispersants, esters different from the dispersants, derivatives of phenol, and sulfonates different from the dispersants. The corrosion inhibitors are advantageously selected from nitrogen compounds such as triazoles and thiadiazoles.

The selection of these ancillary additives, and the adjustment of their respective amounts, is clearly part of the competence of a person skilled in the art.

Finally, in the context of the present invention and in the case of distillation, the monomer composition and/or the polymerization-inhibiting composition or the anti-fouling composition in accordance with the invention may be introduced directly into the distillation column. The purification process, and particularly distillation process, may more particularly be a continuous process. In that case, the supplying of the monomer composition and of the polymerization-inhibiting composition or the anti-fouling composition takes place continuously. The point at which the monomer composition is introduced may vary according to the apparatus used. The selection of these parameters and of the apparatus is part of the general knowledge of a person skilled in the art.

When the polymerization-inhibiting composition or the anti-fouling composition in accordance with the present invention is used in the context of preparation of an ethylenically unsaturated monomer comprising at least one heteroatom, the polymerization-inhibiting composition or the anti-fouling composition may be removed at the end of the process by distillation, washing with sodium hydroxide solution, liquid-liquid extraction, or else by adsorption, which are conventional to a skilled person.

The invention will now be further described in examples. The examples are given by way of illustration and are not intended to limit the specification or the claims in any manner.

The inhibitors and dispersants used in the examples are as follows:
HQ—Hydroquinone flake sold by Solvay,
PTZ—Phenothiazine prill sold by Solvay,
CDBC—Copper dibutyldithiocarbamate powder sold by Solvay
MeHQ—Monomethyl hydroquinone ether flakes sold by Solvay
CuCO₃·Cu(OH)₂—Cupric carbonate basic powder sold by Sinopharm.

### Example 1

The effectiveness of several inhibitor compositions were validated in a dynamic inhibition test, which was a reflux distillation test simulating a step of distillative purification of acrylic acid, as for example disclosed in US 2004/0225151A1 and EP 0695736A1.

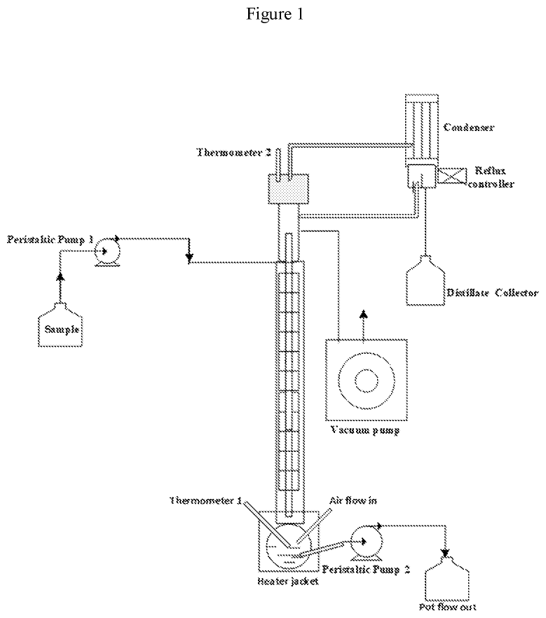

The distillation apparatus used in the dynamic inhibition test comprises a 1000 ml round-bottom flask in glass (hereinafter also referred to as “distillation pot” or “pot”), surmounted by a double-wall refrigerating column (hereinafter also referred to as “distillation column” or “column”) with a height of 33 cm with 10 trays, and a reflux apparatus and a condenser, as shown in FIG. 1.

In the examples, a lab blended sample comprising 62 wt % of acrylic acid, 31.5 wt % of toluene, 3.4 wt % of acetic acid, 3 wt % of water, 0.1% of maleic acid and different inhibitors showed in table 2 was used to mimic the crude aqueous acrylic acid to be purified by the distillation.

Table 1 shows the test conditions of the dynamic inhibition test.

TABLE 1
Dynamic inhibition test conditions.

| Pot setup temperature | 90° | C. |
|---|---|---|
| Air flow rate | 50 | ml/min |
| Reflux ratio | 7 | |
| Column pressure | 240 | mbar |
| Overhead feed rate | 300 | g/hr |
| Top outlet rate | 110 | g/hr |
| Bottom outlet rate | 190 | g/hr |

The dynamic inhibition test comprises the following steps:
1) 500 g of the lab blended sample is prepared, with the respective inhibitor composition added into the sample; 300 g of the sample is added into the flask, and 200 g of the sample is fed overhead by a peristaltic pump into the column.
2) After the 200 g of the lab blended sample is completely added to the flask, pull vacuum at 240 mbar, start heating the flask (to 90° C.) and record the time at which the heating is started. At the same time, start air flow at 50 ml/min to the lab blended sample in the flask.
3) Distillation begins when the temperature reaches optimum settings, at the same time, use the reflux controller to make sure the top outlet rate is at 110 g/hr, meanwhile start the overhead feed peristaltic pump 1 to make sure the feed speed is at 300 g/hr, and the other peristaltic pump 2 draws the concentrated AA in the flask out at a flow rate of 190 g/hr.
4) Watch the top of the column, the condenser and the trays of the column for polymer build-up once every 15 minutes. When polymer is observed on a tray, stop the experiment and record the time, calculate the “inhibition time” which is from start heating to polymer observed on the tray, this time is recorded in table 2 as “inhibition time”.

Following the dynamic inhibition test, several inhibitor compositions were tested to identify the respective inhibition time. Table 2 reports the compositions, dosages and inhibition times of the inhibitor compositions.

TABLE 2
comparison for effectiveness of several inhibitor compositions

| | | | | | | | | Total | |
|---|---|---|---|---|---|---|---|---|---|
| | | | Inhibitors dosage, ppm, relative to | | | | | dosage of | Inhibition |
| Example | | Inhibitor | the weight of the lab blended sample | | | | | inhibitors, | time, |
| No. | | composition | HQ | PTZ | CDBC | Cu* | MeHQ | ppm | hrs |
| comparative | 1.1.1 | HQ | 104.0 | | | | | 104.0 | 4 |
| examples | 1.1.2 | PTZ | | 104.0 | | | | 104.0 | 3.5 |
| | 1.1.3 | HQ/PTZ | 64.0 | 40.0 | | | | 104.0 | 5.5 |
| | 1.1.4 | HQ/PTZ | 34.0 | 70.0 | | | | 104.0 | 6 |
| | 1.1.5 | HQ/PTZ | 52.0 | 52.0 | | | | 104.0 | 6 |
| | 1.1.6 | HQ/PTZ | 57.0 | 57.0 | | | | 114.0 | 6.5 |
| | 1.1.7 | HQ/PTZ | 62.0 | 62.0 | | | | 124.0 | 7 |
| | 1.1.8 | HQ/PTZ | 75.0 | 56.0 | | | | 131.0 | 8 |
| inventive | 1.2 | HQ/PTZ/CDBC | 52.0 | 52.0 | 10.0 | | | 114.0 | 5 |
| examples | 1.3 | HQ/PTZ/MEHQ | 70.0 | 50.0 | | | 10.0 | 130.0 | 9 |
| | 1.4 | HQ/PTZ/Cu/MeHQ | 42.0 | 42.0 | | 10.0 | 20.0 | 114.0 | 12 |
| | 1.5 | HQ/PTZ/Cu/MeHQ | 52.0 | 52.0 | | 10.0 | 10.0 | 124.0 | 14.5 |
| | 1.6 | HQ/PTZ/Cu | 57.0 | 57.0 | | 10.0 | | 124.0 | 14 |
| | 1.7 | HQ/Cu | 114 | | | 10 | | 124.0 | 9.5 |
| | 1.8 | PTZ/Cu | | 114 | | 10 | | 124.0 | 8.5 |

*Cu represents CuCO3•Cu(OH)2.

As can be seen, the inventive inhibitor compositions generally achieve better polymerization inhibition effects, as is evidenced by inhibition time in hours, than comparative inhibitors HQ and/or PTZ, which are conventional in the art, at same total dosages of inhibitors. The combinational use of MeHQ and/or basic copper carbonate with HQ and/or PTZ is therefore demonstrated to achieve synergistic effects with conventional inhibitors HQ and/or PTZ.

### Example 2

The same dynamic inhibition test in the same distillation apparatus as in example 1 was repeated under the same test conditions, to test the polymerization inhibition and anti-fouling effects of several anti-fouling compositions as reported in table 3. The only difference was, the distillation was continued in the present example for 10 hours, i.e., the distillation continued after observation of polymer in pot or on trays.

Each of the compositions comprised 57 ppm of HQ, 57 ppm of PTZ, 10 ppm of basic copper carbonate and 25 ppm of active contents of dispersants, relative to the weight of the lab blended sample, except for example 2.1, which does not comprise any dispersant.

TABLE 3
Dynamic test results of example 2

| | | | Weight of | |
|---|---|---|---|---|
| | | | polymer in | Polymer |
| Ex. | Dispersant active | Dispersant | pot, g | forming in |
| No. | compound | Type | (run 10 hrs) | pot, hrs |
| 2.1 | / | / | 3.5 | 1.0 |
| 2.2 | Ammonium salt of alkyl | alkyl ether | 1.7 | 0.9 |
| | ether sulfate Octyl & Decyl | sulfates | | |
| 2.3 | Ammonium salt of alkyl | alkyl ether | 0.83 | 0.8 |
| | ether sulfate with 20EO | sulfates | | |
| | (trade name: ABEX 2115) | | | |
| 2.4 | Sodium C14-C16 olefin | sulfonates | 0.57 | 1.3 |
| | sulphonate | | | |
| 2.5 | Disodium ethoxylated | Half ester of | 0.7 | 1.2 |
| | alcohol | sulfosuccinic | | |
| | [C10-C12] half ester of | acid | | |
| | sulfosuccinic acid | | | |
| 2.6 | Sodium dodecyl benzene | sulfonates | 1.0 | 1.2 |
| | sulphonate | | | |
| 2.7 | Sodium Lauryl Sulphate | sulfates | 0.37 | 1.0 |
| 2.8 | Sodium laureth sulphate | sulfates | 0.53 | 1.0 |
| | with 4EO | | | |
| 2.9 | Sodium laureth sulphate | sulfates | 1.23 | 1.0 |
| | with 12EO | | | |
| 2.10 | C10-C16 Alkoxy Sodium | sulfates | 0.6 | 1.0 |
| | Ethoxy Sulfate with 30EO | | | |
| 2.11 | Tristyrylphenol Ethoxylate | Polyoxyethylenes | 3.4 | 0.9 |
| | with 10 EO | | | |
| 2.12 | Octylphenol Ethoxylate (30 | Polyoxyethylenes | 0.92 | 0.8 |
| | EO) | | | |
| 2.13 | Sorbitan Monooleate | sorbitan ester | 0.63 | 1.0 |
| 2.14 | Block copolymer of | Polyoxyethylenes | 1.0 | 0.9 |
| | propylene oxide and | | | |
| | ethylene oxide, containing | | | |
| | 40% w/w ethylene oxide | | | |
| 2.15 | Ethoxylated Sorbitan | ethoxylated | 0.86 | 1.0 |
| | Monooleate with 20 EO | sorbitan ester | | |
| 2.16 | C12-C14 Alcohol | Long carbon chain | 1.68 | 1.0 |
| | Ethoxylate (4EO) | fatty alcohol | | |
| | | polyoxyethylene | | |
| | | ether | | |
| 2.17 | Block copolymer of | Polyoxyethylenes | 0.66 | 1.0 W |
| | propylene oxide and | | | |
| | ethylene oxide, containing | | | |
| | 20% w/w ethylene oxide | | | |

All of these dispersants reported in table 3 show a decrease in the weight of polymer formation in the pot following 10 hours of distillation. Examples 2.4, 2.5 and 2.6 also show an unexpected result of the dispersant increasing the time until visible formation of polymer in the pot compared to the Example 2.1 formulation that contains no dispersant.

### Example 3

Example 2 was repeated with a different series of anti-fouling compositions, as reported in table 4, comprising 55 ppm of HQ, 34 ppm of PTZ, 5 ppm of CDBC, 10 ppm of basic copper carbonate and 25 ppm of active contents of dispersants, relative to the weight of the lab blended sample, except for example 3.1, which does not comprise any dispersant.

TABLE 4
Dynamic test results of example 3

| | | Weight of | | | |
|---|---|---|---|---|---|
| | | polymer in | | Polymer | Polymer |
| Ex. | | pot, g | | forming in | forming on |
| No. | Dispersant active compound | (run 10 hours) | | pot, hrs | trays, hrs |
| 3.1 | / | 8.3 | g | 1 | 4.5 |
| 3.2 | Ammonium salt of alkyl ether | 0.7 | g | 1 | 5 |
| | sulfate | | | | |
| | Octyl & Decyl | | | | |
| 3.3 | Sodium C14-C16 olefin | 0.02 | g | 5 | 7 |
| | sulphonate | | | | |
| 3.4 | Disodium ethoxylated alcohol | 0 | g | 4 | 6 |
| | [C10-C12] half ester of | | | | |
| | sulfosuccinic acid | | | | |
| 3.5 | Sodium dodecyl benzene | 0 | g | 5 | 7 |
| | sulphonate | | | | |
| 3.6 | Sodium lauryl Sulphate | 0.03 | g | 2 | 7 |
| 3.7 | Sodium laureth sulphate with | 0.02 | g | 3 | 6 |
| | 4EO | | | | |
| 3.8 | Sodium laureth sulphate with | 0.16 | g | 2 | 6 |
| | 12EO | | | | |
| 3.9 | C10-C16 Alkoxy Sodium Ethoxy | 0.25 | g | 4 | 6.5 |
| | Sulfate with 30EO | | | | |
| 3.10 | Alkyl polyethylene glycol ether | 1.2 | g | 0.9 | 4 |
| | with 11 mole ethylene oxide, | | | | |
| | Branch, C11, 11EO | | | | |
| 3.11 | Block copolymer of propylene | 0.2 | g | 1.5 | 6 |
| | oxide and ethylene oxide, | | | | |
| | containing 20% w/w ethylene | | | | |
| | oxide | | | | |
| 3.12 | Tristyryl phenol ethoxylate with | 1.34 | g | 1 | 4 |
| | 25 EO | | | | |

| | | | | |
|---|---|---|---|---|
| 3.13 | Octylphenol Ethoxylate (30 EO) | 0.21 | 2.5 | 5 |
| 3.14 | Sorbitan Monooleate | 0.23 | 2 | 5 |
| 3.15 | PEG 30 Castor Oil | 0.21 | 2.5 | 5 |
| 3.16 | Block copolymer of propylene | 0.23 | 2 | 5 |
| | oxide and ethylene oxide, | | | |
| | containing 40% w/w ethylene | | | |
| | oxide | | | |
| 3.17 | Ethoxylated Sorbitan Monooleate | 10.12 | 2 | 5 |
| | with 20 EO | | | |
| 3.18 | C12-C14 Alcohol Ethoxylate (4 | 0.16 | 2 | 5 |
| | EO) | | | |
| 3.19 | Sodium Acrylate Copolymer | 0.36 | 2 | 5 |
| | (Mw = 4000) | | | |

In Table 4, again, all of the dispersants show a reduction of polymer formation in the pot. Examples 3.3 to 3.9, 3.11 and 3.13 to 3.19 all show the unexpected result of the dispersant increasing the time until visible formation of polymer in the pot compared to the Example 1 formulation that contains no dispersant. Examples 3.2 to 3.9, 3.11 and 3.13 to 3.19 all show the surprising result of also increasing the time to polymer formation in the distillation column as compared to the Example 1 formulation that contains no dispersant.

### Example 4

Example 2 was repeated with a different series of anti-fouling compositions, as reported in table 5, comprising 59 ppm of HQ, 45 ppm of PTZ and 25 ppm of active content of dispersants, relative to the weight of the lab blended sample, except for example 4.1 which does not comprise any dispersant.

TABLE 5
Dynamic test results of example 4

| | | Weight of | | Weight of | | | |
|---|---|---|---|---|---|---|---|
| | | polymer in | | polymer on | | Polymer | Polymer |
| Ex. | | pot, g | | trays, g | | forming in | forming on |
| No. | Dispersant active compound | (run 10 hours) | | (run 10 hours) | | pot, hrs | trays, hrs |
| 4.1 | / | 5 | g | 0.16 | g | 0.8 | 5.5 |
| 4.2 | Tristyrylphenol Ethoxylate | 3.7 | g | 0.13 | g | 1 | 6.5 |
| | with 10 EO | | | | | | |
| 4.3 | Block copolymer of | 3 | g | 0.15 | g | 1 | 6.5 |
| | propylene oxide and ethylene | | | | | | |
| | oxide, containing 20% w/w | | | | | | |
| | ethylene oxide | | | | | | |
| 4.4 | Sodium Lauryl Sulfate | 2 | g | 0.03 | g | 1 | 7 |
| 4.5 | Ammonium salt of alkyl ether | 2.9 | g | 0.02 | g | 1 | 7 |
| | sulfate Octyl & Decyl | | | | | | |
| 4.6 | Sorbitan Monooleate | 2.5 | g | 0.03 | g | 1 | 7.5 |
| 4.7 | C12-C14 Alcohol | 5 | g | 0.05 | g | 1 | 7.5 |
| | Ethoxylate (4 EO) | | | | | | |

As shown in table 5, all dispersants demonstrated better effects with regard to the weight of polymer in the pot and/or in the trays, or the time period to avoid formation of polymer in the pot and/or in the trays, than the comparative example 4.1 with no dispersant.

1. An anti-fouling composition comprising at least one polymerization inhibitor and at least one dispersant selected from the group consisting of alkyl ether sulfates, sulfonates, half esters of sulfosuccinic acid, sulfates, polyoxyethylenes, sorbitan and ethoxylated sorbitan esters, fatty alcohol ethoxylates and ethoxylate/propoxylate block copolymers.

2. The anti-fouling composition of claim 1, wherein the at least one polymerization inhibitor is selected from the group consisting of hydroquinone (HQ), phenothiazine (PTZ), monomethyl hydroquinone ether (MEHQ), TEMPO and its derivatives, and copper salts.

3. The anti-fouling composition of claim 2, wherein the at least one polymerization inhibitor comprises or consists of hydroquinone (HQ) and phenothiazine (PTZ).

4. The anti-fouling composition of claim 3, wherein the weight ratio of HQ:PTZ is in the range of (0.4-2.5):1.

5. The anti-fouling composition of claim 2, wherein the at least one polymerization inhibitor is selected from the group consisting of TEMPO and its derivatives.

6. The anti-fouling composition of claim 3, wherein the at least one polymerization inhibitor further comprises:
monomethyl hydroquinone ether (MEHQ), in an amount of 0.1 wt %-40 wt %, relative to the total weight of the at least one polymerization inhibitor; and/or
at least one copper salt, in an amount of 1 wt %-30 wt relative to the total weight of the at least one polymerization inhibitor.

7. The anti-fouling composition of claim 2, wherein the copper salt is selected from the group consisting of (basic) copper sulfate, copper acetate, copper nitrate, (basic) copper carbonate, copper chloride, copper naphthenate, copper benzoate, copper stearate, copper acrylate and CDBC (Copper dibutyldithiocarbamate).

8. The anti-fouling composition of claim 1, wherein the at least one dispersant is selected from the group consisting of sulfonates, half esters of sulfosuccinic acid, sulfates, sorbitan and ethoxylated sorbitan esters, fatty alcohol ethoxylates and ethoxylate/propoxylate block copolymers.

9. The anti-fouling composition of claim 1, wherein the at least one dispersant comprises 1 wt %-50 wt % of the total weight of the anti-fouling composition.

10. The anti-fouling composition of claim 1 comprising:
a first portion comprising the at least one dispersant and optionally MEHQ,
a second portion comprising HQ and optionally (basic) copper carbonate, and
a third portion comprising PTZ and optionally CDBC.

11. The anti-fouling composition of claim 1 comprising:
a first portion comprising the at least one dispersant and optionally MEHQ, and
a fourth portion comprising TEMPO and/or its derivatives, and optionally at least one selected from the group consisting of HQ, PTZ, (basic) copper carbonate and CDBC.

12. The anti-fouling composition of claim 10, wherein the first portion comprises 15-99 wt % of the at least one dispersant and 1-10 wt % of MEHQ.

13. The anti-fouling composition of claim 1 which is used in a monomer composition, wherein the anti-fouling composition is present in the monomer composition in a range of 10-5000 ppm by weight.

14. The anti-fouling composition of claim 13, wherein the monomer composition comprises at least one ethylenically unsaturated monomer comprising at least one heteroatom.

15. The anti-fouling composition of claim 14, wherein the at least one ethylenically unsaturated monomer comprising at least one heteroatom is selected from the group consisting of halogenated unsaturated monomers, acrylic monomers, unsaturated acrylic resins, unsaturated amides, unsaturated ethers, vinylpyridines and vinyl acetate.

16. The anti-fouling composition of claim 15, wherein the at least one ethylenically unsaturated monomers comprising at least one heteroatom is selected from the group consisting of acrylic acid, methacrylic acid, methyl acrylate, ethyl acrylate, methyl methacrylate, ethyl methacrylate, and acrylonitrile.

17. A method for purifying a monomer composition, which comprises at least one ethylenically unsaturated monomer comprising at least one heteroatom, comprising a distillation step of the monomer composition in the presence of the anti-fouling composition of claim 1.

18. A method to limit and/or prevent fouling of industrial equipment used during reaction, distillation, purification, storage, transportation and/or handling comprising, adding the anti-fouling composition of claim 1 to the industrial equipment used during reaction, distillation, purification, storage, transportation and/or handling.

19. The anti-fouling composition of claim 10 consisting of:
a first portion comprising the at least one dispersant and optionally MEHQ,
a second portion comprising HQ and optionally (basic) copper carbonate, and
a third portion comprising PTZ and optionally CDBC.

20. The anti-fouling composition of claim 11, consisting of:
a first portion comprising the at least one dispersant and optionally MEHQ, and
a fourth portion comprising TEMPO and/or its derivatives, and optionally at least one selected from the group consisting of HQ, PTZ, (basic) copper carbonate and CDBC.
